(19) United States
(12) Patent Application Publication
(10) Pub. No.: US 2005/0288491 A1
(43) Pub. Date: Dec. 29, 2005

(54) Super-humanized antibodies against respiratory syncytial virus

(76) Inventors: David S. Wilson, Mountain View, CA (US); Steffen Nock, Redwood City, CA (US); James W. Larrick, Woodside, CA (US)

Correspondence Address: Birgit Millauer;DORSEY & WHITNEY LLP, Intellectual Property Department, 555 California Street, Suite 1000, San Francisco, CA, 94104-1513, US

(21) Appl. No.: 11/061,848

(22) Filed: Feb. 17, 2005

Related U.S. Application Data
(60) Provisional application No. 60/545,011, filed on Feb. 17, 2004.

(51) Int. Cl. A61K 39/42; C07K 16/44

(52) U.S. Cl. 530/388.15; 530/388.3

(57) ABSTRACT

Disclosed herein are humanized antibodies that bind to an epitope on the F protein of respiratory syncytial virus. The humanized antibodies were designed by comparing the canonical CDR structure types of the CDRs from a non-human antibody (HNK20) to the canonical CDR structure types found in the human antibody germline sequences as the basis for selecting human variable region frameworks in a method denoted “super-humanization.” Human antibody variable regions having the same or similar canonical CDR structure types as the non-human CDR provided a subset of candidate sequences from which to select the human frameworks. Chimeric variable regions were made comprising the non-human CDRs grafted in corresponding locations into the human frameworks from the candidate human variable regions. Several humanized antibodies that bind the same antigen as HNK20 and that have low immunogenicity were thereby designed, including examples where the framework sequences have less than 65% amino acid identity to the non-human frameworks.

### CROSS-REFERENCE TO RELATED APPLICATION(S)

This application claims priority to U.S. provisional patent application No. 60/545,011 filed Feb. 17, 2004.

### TECHNICAL FIELD

The disclosure relates to humanized antibodies that bind to respiratory syncytial virus and to methods of selecting appropriate human antibody framework sequences for performing the humanization, and more particularly to comparing canonical CDR structure types between non-human and human antibody genes as the basis for selecting appropriate human framework sequences for performing grafting of CDRs to make the humanized anti RSV antibody.

### BACKGROUND

Respiratory syncytial virus (RSV) is a virus that causes infection of the lungs and breathing passages. It can infect the same person several times during a lifetime, causing more severe illnesses (like pneumonia) in infancy, but only a common cold in adulthood. After each RSV infection, the body forms some immunity to the virus, but that immunity is never complete. Re-infections occur, but they usually are less severe than earlier RSV attacks. RSV passes from person to person through infected nasal and oral fluids. It can enter the body when eyes or nose are touched. Primary infection with respiratory syncytial virus occurs during the first two years of life, causing self-limited disease of the upper or lower respiratory tract. In some cases, infection of the lower respiratory tract causes severe bronchitolitis or pneumonia. Those at highest risk for lower respiratory tract disease include infants and children with bronchopulmonary dysplasia, congenital heart disease, and immunodeficiency disorders. Each year, RSV infections lead to more than 125,000 hospitalizations and about 2,500 deaths.

Since efforts to develop a safe and effective vaccine against RSV have not yet succeeded, passive immunization with polyclonal or monoclonal antibodies has been used as an alternative strategy for prophylaxis (Anderson L J, Bingham P, Hierholzer J C. (1988). Neutralization of respiratory syncytial virus by individual and mixtures of F and G protein monoclonal antibodies. J Virol6 2(11):4232-8). Medimmune's Synagis, a humanized monoclonal antibody, is the only monoclonal prophylactic agent approved today. In a phase III clinical trial, the Synagis group had 45-percent fewer hospitalizations due to RSV than the placebo group in a phase III trial.

RSV causes serious lower respiratory tract disease. RSV is responsible for nearly 50% of cases of children hospitalized for bronchiolitis and 25% of children with pneumonia.

RSV is an enveloped negative strand RNA virus of the genus Pneumovirus, Paramyxoviridae family. There are two viral surface proteins, F and G, both of which are glycosylated. The F protein (68 kD) mediates fusion with target cells. Antibodies against the F protein have been described, for example in U.S. Pat. No. 5,534,411 and No. 6,258,529, incorporated herein in entirety.

Antibodies are natural proteins that the vertebrate immune system forms in response to foreign substances (antigens), primarily for defense against infection. For over a century, antibodies have been induced in animals under artificial conditions and harvested for use in therapy or diagnosis of disease conditions, or for biological research. Each individual antibody-producing cell produces a single type of antibody with a chemically defined composition, however, antibodies obtained directly from animal serum in response to antigen inoculation actually comprise an ensemble of non-identical molecules (i.e., polyclonal antibodies) made from an ensemble of individual antibody-producing cells.

Hybridoma technology provided a method to propagate a single antibody-producing cell for an indefinite number of generations with a screening method to identify clones of cells producing an antibody that would react with a particular antigen. Development of this technology allowed production in unlimited quantities of structurally identical antibodies with essentially any desired antigenic specificity. Such antibodies are commonly called monoclonal antibodies, and most originate from rodents (usually mice, rats or rabbits), but human monoclonal antibodies have also been produced. Sequencing of monoclonal antibody genes allowed the primary amino acid structure of the antibody to be defined.

The advent of recombinant DNA methodology enabled structural engineering of antibody genes and production of modified antibody molecules with properties not obtainable by hybridoma technology. In the therapeutic arena, one aim of this methodology has been to reduce the immunogenicity in humans of rodent monoclonal antibodies by modifying their primary amino acid structure. Reduction of the immunogenicity of therapeutic antibodies is desirable because induction of an immune response can cause a spectrum of adverse effects in a patient, ranging from accelerated elimination of the therapeutic antibody, with consequent loss of efficacy, to fatal anaphylaxis at the most extreme.

One strategy to reduce immunogenicity of foreign monoclonal antibodies has been to replace the light and heavy chain constant domains of the monoclonal antibody with analogous domains of human origin, leaving the variable region domains of the foreign antibody intact. The variable region domains of the light and heavy chains are responsible for the interaction between the antibody and the antigen. The joining domains connecting variable domains to constant domains are situated in a region remote from the site of antigen-binding, therefore, the joining domains between variable and constant domains generally do not interfere with antigen-binding. Chimeric antibody molecules having mouse variable domains joined to human constant domains usually bind antigen with the same affinity constant as the mouse antibody from which the chimeric antibody was derived. Such chimeric antibodies are less immunogenic in humans than their fully murine counterparts. Nevertheless, antibodies that preserve entire murine variable domains tend to provoke immune responses in a substantial fraction of patients. For example, INFLIXIMAB™, a widely prescribed chimeric antibody that is considered safe, induced a human anti-chimeric antibody response in 7 out of 47 Crohns Disease patients. (Rutgeerts, P., et al (1999) Efficacy and safety of retreatment with anti-tumor necrosis factor antibody (INFLIXIMAB) to maintain remission in Crohn's disease. Gastroenterology 117, 761-769).

That humans would mount an immune response to whole murine variable domains was predictable. Thus, efforts to obtain variable domains with more human character had begun even before clinical trials of such standard chimeric antibodies had been reported. One category of methods frequently referred to as “humanizing” aims to convert the variable domains of murine monoclonal antibodies to a more human form by recombinantly constructing an antibody variable domain having both mouse and human character. Humanizing strategies are based on several consensual understandings of antibody structure data. First, variable domains contain contiguous tracts of peptide sequence that are conserved within a species, but which differ between evolutionarily remote species, such as mice and humans. Second, other contiguous tracts are not conserved within a species, but even differ even between antibody producing cells within the same individual. Third, contacts between antibody and antigen occur principally through the non-conserved regions of the variable domain. Fourth, the molecular architecture of antibody variable domains is sufficiently similar across species that correspondent amino acid residue positions between species may be identified based on position alone, without experimental data.

Antibody humanization strategies share the premise that replacement of amino acid residues that are characteristic of murine sequences with residues found in the correspondent positions of human antibodies will reduce the immunogenicity in humans of the resulting antibody. However, replacement of sequences between species usually results in reduction of the binding affinity of an antibody to its antigen. The art of humanization therefore lies in balancing replacement of the original murine sequence to reduce immunogenicity with the need for the humanized molecule to retain sufficient antigen binding affinity to be therapeutically useful. This balance has been struck using two approaches.

Wu and Kabat pioneered the alignment of antibody peptide sequences, and their contributions in this regard were several-fold: first, through study of sequence similarities between variable domains, they identified correspondent residues that to a greater or lesser extent were homologous across all antibodies in all vertebrate species, inasmuch as they adopted similar three-dimensional structures, played similar functional roles, interacted similarly with neighboring residues, and existed in similar chemical environments. Second, they devised a peptide sequence numbering system in which homologous immunoglobulin residues were assigned the same position number. One skilled in the art can unambiguously assign what is now commonly called “Kabat numbering” to any variable domain sequence, without reliance on any experimental data beyond the sequence itself. Third, for each Kabat-numbered sequence position, Kabat and Wu calculated variability, by which is meant the finding of few or many possible amino acids when variable domain sequences are aligned. They identified three contiguous regions of high variability embedded within four less variable contiguous regions. Other workers had previously noted variability approximately in these regions (hypervariable regions) and posited that the highly variable regions represented amino acid residues used for antigen-binding. Kabat and Wu formally demarcated residues constituting these variable tracts, and designated these “complementarity determining regions” (CDRs), referring to chemical complementarity between antibody and antigen. A role in three-dimensional folding of the variable domain, but not in antigen recognition, was ascribed to the remaining less-variable regions, which are now termed “framework regions”. Fourth, Kabat and Wu established a public database of antibody peptide and nucleic acid sequences, which continues to be maintained and is well known to those skilled in the art.

The humanization method disclosed by Winter and Jones using the Kabat classification results in a chimeric antibody comprising CDRs from one antibody and framework regions from another antibody that differs in species origin, specificity, subclass, or other characteristics (Jones, P. T., Dear, P. H., Foote, J., Neuberger, M. S. & Winter, G. (1986) Replacing the complementarity-determining regions in a human antibody with those from a mouse. Nature 321, 522-525).

However, no particular sequences or properties were ascribed to the framework regions, indeed, Winter taught that any set of frameworks could be combined with any set of CDRs. Framework sequences have since been recognized as being important for conferring the three dimensional structure of an antibody variable region necessary retain good antigen binding. Thus, the general humanizing methods described by Winter and Jones have the disadvantage of frequently leading to inactive antibodies because these references do not provide information needed to rationally select among the many possible human framework sequences, those most likely to support antigen binding required by a particular CDR region from a non-human antibody. Subsequent developments in the field have been refinements within the scope of Winter to deal with loss of avidity for antigen observed with some humanized antibodies relative to the avidity of the corresponding mouse antibodies. (Avidity is a quantitative measure of partitioning of an antibody, in the presence of antigen under conditions approximating chemical equilibrium, between free and antigen-bound forms. For reactions in solution not subject to multivalent binding effects, avidity is the same as affinity, the biochemical equilibrium constant.).

U.S. Pat. No. 5,693,761 to Queen et al, discloses one refinement on Winter for humanizing antibodies, and is based on the premise that ascribes avidity loss to problems in the structural motifs in the humanized framework which, because of steric or other chemical incompatibility, interfere with the folding of the CDRs into the binding-capable conformation found in the mouse antibody. To address this problem, Queen teaches using human framework sequences closely homologous in linear peptide sequence to framework sequences of the mouse antibody to be humanized. Accordingly, the methods of Queen focus on comparing framework sequences between species. Typically, all available human variable domain sequences are compared to a particular mouse sequence and the percentage identity between correspondent framework residues is calculated. The human variable domain with the highest percentage is selected to provide the framework sequences for the humanizing project. Queen also teaches that it is important to retain in the humanized framework certain amino acid residues from the mouse framework critical for supporting the CDRs in a binding-capable conformation. Potential criticality is assessed from molecular models. Candidate residues for retention are typically those adjacent in linear sequence to a CDR or physically within 6 angstroms of any CDR residue.

There is therefore, a need in the art for methods of humanizing antibodies that reliably identify suitable human framework sequences to support non-human CDR regions and to provide humanized antibodies that retain high antigen binding with low immunogenicity in humans, without the need for direct comparison of framework sequences, without need for determining critically important amino acid residues in the framework, and without need for multiple iterations of construction to obtain humanized antibodies with suitable therapeutic properties. This need can be applied to humanized antibodies against respiratory syncytial virus (RSV).

### SUMMARY

The present invention meets this need by providing methods for making humanized antibodies against respiratory syncytial virus of high affinity and low immunogenicity without need for comparing framework sequences between non-human and human antibodies and also provides humanized antibodies made thereby. Rather than relying on human framework sequences as the point of analysis, the methods provided herein rely on comparing canonical CDR structure types of the non-human antibody that binds respiratory syncytial virus to CDR structure types of human antibody variable region sequences, particularly as from such sequences present in the human germline sequence, to identify candidate human antibody sequences from which to obtain appropriate human frameworks.

More particularly, there is provided several candidate human antibody variable region heavy and light chain framework sequences into which the CDRs of the murine anti-RSV antibody HNK20 may be grafted, and several examples of such CDR-grafted, humanized sequences.

### BRIEF DESCRIPTION OF THE DRAWINGS

FIG. 1. Shows human germline heavy chain variable region-encoded amino acid sequences that may be used for CDR grafting of the HNK20 V_{H} CDRs, aligned to the murine HNK20 V_{H} CDRs.

FIG. 2. Shows an alignment of the murine HNK20 V_{H} CDR3 with human germline J_{H} sequences.

FIG. 3. Shows exemplary embodiments of super-humanized heavy chain variable regions.

FIG. 4. Shows human germline light (kappa) chain variable region-encoded amino acid sequences that may be used for CDR-grafting of the HNK20 Vₖ CDRs, aligned to the murine HNK20 Vₖ CDRs.

FIG. 5. Shows preferred human germline light (kappa) chain variable region-encoded amino acid sequences that may be used for CDR-grafting of the HNK20 Vₖ CDR, aligned to the murine HNK20 Vₖ CDRs.

FIG. 6. Shows an alignment of the murine HNK20 Vₖ CDR3 with the human germline Jₖ sequences.

FIG. 7. Shows exemplary embodiments of super humanized Vₖ variable regions.

### DETAILED DESCRIPTION OF PREFERRED EMBODIMENTS

In the description that follows, citation is made to various references that may assist one of ordinary skill in the art in understanding and practicing the invention to its fullest extent. Therefore, each reference cited in the description that follows is incorporated herein by reference in its entirety. To better aid in understanding various embodiments of the invention it may be helpful to explain the meanings of certain terms used herein.

A “mature antibody gene” is a genetic sequence encoding an immunoglobulin that is expressed, for example, in a lymphocyte such as a B cell, in a hybridoma or in any antibody-producing cell that has undergone a maturation process so that the particular immunoglobulin is expressed. The term includes mature genomic, cDNA or other nucleic acid sequence that encodes such mature genes, which may have been isolated and/or recombinantly engineered for expression in other cell types. Mature antibody genes have undergone various mutations and rearrangements that structurally distinguish them from antibody genes encoded in all cells other than lymphocytes. Mature antibody genes in humans, rodents, and many other mammals are formed by fusion of V and J gene segments in the case of antibody light chains and fusion of V, D, and J gene segments in the case of antibody heavy chains. Many mature antibody genes acquire point mutations subsequent to fusion, some of which increase the affinity of the antibody protein for a specific antigen

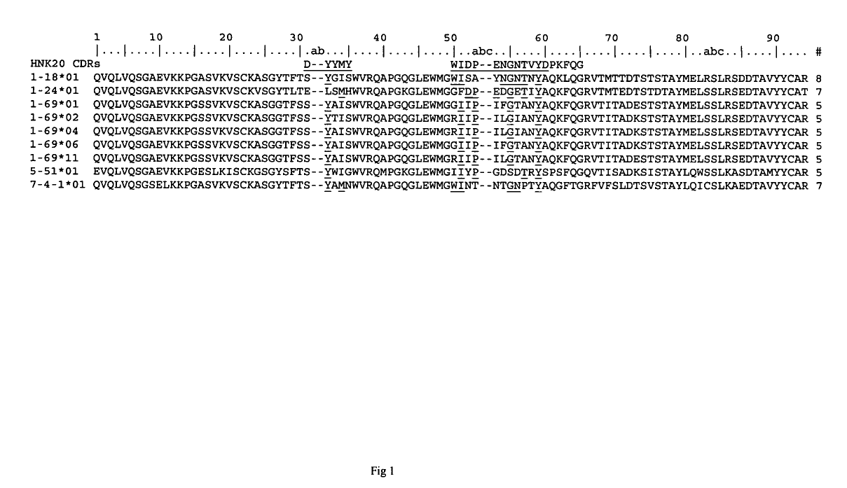

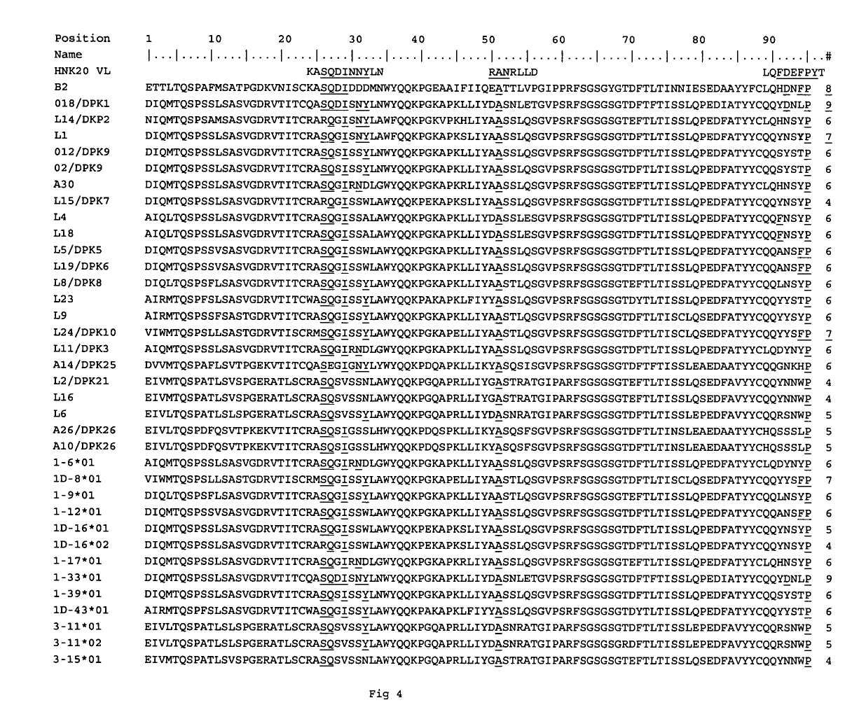

“Germline antibody genes” or gene fragments are immunoglobulin sequences encoded by non-lymphoid cells that have not undergone the maturation process that leads to genetic rearrangement and mutation for expression of a particular immunoglobulin. One of the advantages provided by various embodiments of the present invention stems from the recognition that germline antibody genes are more likely than mature antibody genes to conserve essential amino acid sequence structures characteristic of individuals in the animal species, hence less likely to be recognized as foreign when used therapeutically in that species. FIG. 1 and FIG. 4 show peptide sequences for human germline antibody genes encoding human variable heavy region (V_{H}) and variable light region (Vₖ) antibodies (i.e., immunoglobulins). Each of these list of sequences exemplify a library of human antibody genes, particularly a library of human germline antibody genes.

By “CDR grafting” and grammatical equivalents we mean replacement of part of or all of a CDR in a human variable region with a corresponding CDR of a non-human variable region. Thus, for example, CDR grafting may involve alteration or replacement of some but not all residues of a CDR of a human variable region provided that the result is a grafted CDR that has the same functional characteristics (or recognizes the same antigen) as the corresponding CDR of the non-human variable region.

“A CDR” is the complement determining region within antibody variable sequences. There are three CDRs in each of the variable heavy and variable light sequences designated CDR1, CDR2 and CDR3, for each of the variable regions. The exact boundaries of these CDRs have been defined differently according to different systems, however, all have overlapping residues in what constitute the so called “hypervariable regions” within the variable sequences. The system described by Kabat (Kabat, E. A., Wu, T. T., Perry, H. M., Gottesman, K. S. & Coeller, K. (1991) Sequences of proteins of immunological interest. 5th ed. 1991, Bethesda: U.S. Dept. of Health and Human Services, PHS, NIH; Wu, T. T. & Kabat, E. A. (1970) An analysis of the sequences of the variable regions of Bence Jones proteins and myeloma light chains and their implications for antibody complementarity. J. Exp. Med. 132, 211-250).not only provides an unambiguous residue numbering system applicable to any variable region of an antibody, but also provides precise residue boundaries defining the three CDRs. These CDRs may be referred to as Kabat CDRs. Chothia and coworkers (Chothia, C. & Lesk, A. M. (1987) Canonical structure types for the hypervariable regions of immunoglobulins. J. Mol. Biol. 96, 901-917; Chothia, C., Lesk, A. M., Gherardi, E., Tomlinson, I. M., Walter, G., Marks, J. D., Llewelyn, M. B. & Winter, G. (1992) Structural repertoire of the human V_{H} segments. J. Mol. Biol. 227, 799-817; Tomlinson, I. M., Cox, J. P. L., Gherardi, E., Lesk, A. M. & Chothia, C. (1995) The structural repertoire of the human Vₖ domain. EMBO J. 14, 4628-4638.) found that certain sub portions within Kabat CDRs adopt nearly identical peptide backbone conformations, despite having great diversity at the level of amino acid sequence. These sub portions were designated as L1, L2 and L3 or H1, H2 and H3 where the “L” and the “H” designates the light chain and the heavy chains regions, respectively. These regions may be referred to as Chothia CDRs, which have boundaries that overlap with Kabat CDRs. Table I illustrates the overlap of Chothia and Kabat CDRs according to the residue numbering system of Kabat.

TABLE I

| Chain | CDR | Kabat | Chothia |
|---|---|---|---|
| Light | CDR1 | 24-34 | 26-32 |
| Light | CDR2 | 50-56 | 50-52 |
| Light | CDR3 | 89-96 | 91-96 |
| Heavy | CDR1 | 31-35 | 26-32 |
| Heavy | CDR2 | 50-65 | 52-56 |
| Heavy | CDR3 | 95-102 | not uniquely |
| | | | defined |

Other boundaries defining CDRs overlapping with the Kabat CDRs have been described by Padlan (Padlan, E. O., Abergel, C. & Tipper, J. P. (1995) Identification of specificity-determining residues in antibodies. FASEB J. 9, 133-139.) or MacCallum (MacCallum, R. M., Martin, A. C. R. & Thornton, J. M. (1996) Antibody-antigen interactions: contact analysis and binding site topography. J. Mol. Biol. 262, 732-745.). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although preferred embodiments use Kabat or Chothia-defined CDRs.

“Framework” or “framework sequence” are the remaining sequences of a variable region minus the CDRs. Because the exact definition of a CDR sequence can be determined by different systems, the meaning of a framework sequences is subject to correspondingly different interpretations. To clarify the meaning used herein, a framework sequence means those sequences within the variable region of an antibody other than those defined to be CDR sequences, so that the exact sequence of a framework depends only on how the CDR is defined. For example, the CDRs used in the methods provided herein are usually a subset of what is considered a Kabat CDR, but in the case of CDR1 of heavy chains for example, also includes residues that are classified as framework residues in the Kabat system.

“Canonical CDR structure types” are the structure types designated by Chothia (Chothia, C. & Lesk, A. M. (1987) Canonical structure types for the hypervariable regions of immunoglobulins. J. Mol. Biol. 96, 901-917; Chothia, C., Lesk, A. M., Gherardi, E., Tomlinson, I. M., Walter, G., Marks, J. D., Llewelyn, M. B. & Winter, G. (1992) Structural repertoire of the human V_{H} segments. J. Mol. Biol. 227, 799-817; Tomlinson, I. M., Cox, J. P. L., Gherardi, E., Lesk, A. M. & Chothia, C. (1995) The structural repertoire of the human Vₖ domain. EMBO J. 14, 4628-4638.). Chothia and coworkers found that critical portions of the CDRs of many antibodies adopt nearly identical peptide backbone conformations, despite great diversity at the level of amino acid sequence. Accordingly, Chothia defined for each CDR in each chain one or a few “canonical structures”. Each canonical structure specifies primarily a set of peptide backbone torsion angles for a contiguous segment of amino acid residues forming a loop. The canonical CDR structure types defined by Chothia are listed in Table II.

TABLE II

| | | Canonical |
|---|---|---|
| Chain | CDR | Structure types |
| Kappa | CDR1 | 1-6 |
| Kappa | CDR2 | 1 |
| Kappa | CDR3 | 1-6 |
| Heavy | CDR1 | 1-3 |
| Heavy | CDR2 | 1-4 |
| Lambda | CDR1 | 1-4 |
| Lambda | CDR2 | 1 |
| Lambda | CDR3 | 1-2 |

“Corresponding CDRs” refer relatively to the CDRs between two different variable sequences that correspond in position within the two different variable sequences. Thus, for example, a mouse light chain CDR1 corresponds to a human light chain CDR1, and vice a versa, because each maps to a defined position in a Kabat numbering system, whether or not the actual boundary of the CDR is defined by Kabat, Chothia or some other system. Similarly, “corresponding” residues, sequences or amino acids refer relatively to the residue positions between two different peptide sequences mapped by the Kabat numbering system.

The objective of the methods provided herein, which may be called a CDR-grafting method, is to provide a prescription for arriving at appropriate human framework sequence for humanizing a subject non-human antibody. The methods are based on U.S. Pat. Pub. No. 20030039649, which designates the methods as SUPER-HUMANIZING ANTIBODIES® and to antibodies made thereby as SUPER-HUMANIZED ANTIBODIES.® In all previous CDR-grafting methods, the choice of the humanized framework sequence was based on comparing the human frameworks to the subject (murine) frameworks. In contrast, the basis of the methods for SUPER-HUMANIZING ANTIBODIES® as previously described and as described herein, are to chose the human antibody to provide the humanized framework based on similarity of its CDRs to those of the subject antibody, without regard to comparing the framework sequences between the two antibodies.

The similarity to the subject CDRs of candidate human antibody sequences is assessed for each domain at two levels. Primarily, identical three-dimensional conformations of CDR peptide backbones are sought. Experimentally determined atomic coordinates of the subject CDRs are seldom available, hence three-dimensional similarity is approximated by determining Chothia canonical structure types of the subject CDRs and excluding from further consideration candidates possessing different canonical structures. Secondarily, residue-to-residue homology between subject CDRs and the remaining human candidate CDRs is considered, and the candidate with the highest homology is chosen.

Choosing highest homology is based on various criterion used to rank candidate human variable regions having the same canonical structure as the subject the non-human variable regions. The criterion for ranking members of the selected set may be by amino acid sequence identity or amino acid homology or both. Amino acid identity is simple a score of position by position matches of amino acid residues. Similarity by amino acid homology is position by position similarity in residue structure of character. Homology may be scored, for example, according to the tables and procedures described by Henikoff and Henikoff, (1Amino acid substitution matrices from protein blocks, Proc. Natl. Acad. Sci 89: 10915-10919, 1992) or by the BLOSUM series described by Henikoff and Henikoff (1996).

The steps of the methods are as follows:

Determine the peptide sequences of the heavy and light chain variable domains of the subject antibody that binds to RSV. These can be determined by any of several methods, such as DNA sequencing of the respective genes after conventional cDNA cloning; DNA sequencing of cloning products that have been amplified by the polymerase chain reaction from reverse transcripts or DNA of the subject hybridoma line; or peptide sequencing of a purified antibody protein.

Apply the Kabat numbering system (Kabat, E. A., Wu, T. T., Perry, H. M., Gottesman, K. S. & Coeller, K. (1991) Sequences of proteins of immunological interest. 5th ed. 1991, Bethesda: U.S. Dept. of Health and Human Services, PHS, NIH.) to the heavy and light chain sequences of the subject non-human RSV-binding antibody.

Determine canonical structure types for each of the CDRs of the subject non-human antibody that binds to RSV. This determination is made from examination of the peptide sequence in light of the guidelines discussed in Chothia and Lesk (Chothia, C. & Lesk, A. M. (1987) Canonical structure types for the hypervariable regions of immunoglobulins. J. Mol. Biol. 96, 901-917.), Chothia et al (Chothia, C., Lesk, A. M., Gherardi, E., Tomlinson, I. M., Walter, G., Marks, J. D., Llewelyn, M. B. & Winter, G. (1992) Structural repertoire of the human V_{H} segments. J. Mol. Biol. 227, 799-817.), Tomlinson et al (Tomlinson, I. M., Cox, J. P. L., Gherardi, E., Lesk, A. M. & Chothia, C. (1995) The structural repertoire of the human Vₖ domain. EMBO J. 14, 4628-4638.), MacCallum and colleagues (MacCallum, R. M., Martin, A. C. R. & Thornton, J. M. (1996) Antibody-antigen interactions: contact analysis and binding site topography. J. Mol. Biol. 262, 732-745.), and Al-Lazikani et al (Al-Lazikani B, Lesk A M, Chothia C. (1997). Standard conformations for the canonical structures of immunoglobulins. J Mol. Biol. 273(4):927-48). The salient features of canonical structure determination for each of the CDRs are as follows.

For heavy chain CDR1, three canonical structure types are currently known. Assignment of a new sequence is straightforward because each canonical structure type has a different number of residues. As described in Al-Lazikani et. al (Al-Lazikani B, Lesk A M, Chothia C. (1997). Standard conformations for the canonical structures of immunoglobulins. J Mol. Biol. 273(4):927-48), when Kabat numbering is assigned to the sequence, the numbering for residues 31-35 will be as follows for the respective canonical structures.
Canonical structure type 1: 31, 32, 33, 34, 35.
Canonical structure type 2: 31, 32, 33, 34, 35, 35a.
Canonical structure type 3: 31, 32, 33, 34, 35, 35a, 35b.

For heavy chain CDR2, four canonical structure types are currently known. Several have unique numbers of residues, and are easily distinguished from their unique Kabat numbering of positions 52-56, viz.:
Canonical structure type 1: 52, 53, 54, 55, 56.
Canonical structure type 4: 52, 52a, 52b, 52c, 53, 54, 55, 56. Canonical structure types 2 and 3 for heavy chain CDR2 have equal numbers of residues, hence must be distinguished by clues within their sequence, as discussed by Chothia et al (Chothia, C., Lesk, A. M., Gherardi, E., Tomlinson, I. M., Walter, G., Marks, J. D., Llewelyn, M. B. & Winter, G. (1992) Structural repertoire of the human VH segments. J. Mol. Biol. 227, 799-817). The Kabat numbering of the segment containing these clues is: 52, 52a, 53, 54, 55. Canonical structure type 2 has Pro or Ser at position 52a and Gly or Ser at position 55, with no restriction at the other positions. Canonical structure type 3 has Gly, Ser, Asn, or Asp at position 54, with no restriction at the other positions. These criteria are sufficient to resolve the correct assignment in most cases. Additionally framework residue 71 is commonly Ala, Val, Leu, Ile, or Thr for canonical structure type 2 and commonly Arg for canonical structure type 3.

Heavy chain CDR3 is the most diverse of all the CDRs. It is generated by genetic processes, some of a random nature, unique to lymphocytes. Consequently, canonical structures for CDR3 have been difficult to predict. In any case, human germline V gene segments do not encode any part of CDR3; because the V gene segments end at Kabat position 94, whereas positions 95 to 102 encode CDR3. For these reasons, canonical structures of CDR3 are not considered for choosing candidate human sequences.

For light chain CDR1, six canonical structure types are currently known for CDR1 in kappa chains. Each canonical structure type has a different number of residues, hence assignment of a canonical structure type to a new sequence is apparent from the Kabat numbering of residue positions 27-31.
Canonical structure type 1: 27, 29, 30, 31.
Canonical structure type 2: 27, 28, 29, 30, 31.
Canonical structure type 3: 27, 27a, 27b, 27c, 27d, 27e, 27f, 28, 29, 30, 31.
Canonical structure type 4: 27, 27a, 27b, 27c, 27d, 27e, 28, 29, 30, 31.
Canonical structure type 5: 27, 27a, 27b, 27c, 27d, 28, 29, 30, 31. Canonical structure type 6: 27, 27a, 28, 29, 30, 31.

For light chain CDR2, only a single canonical structure type is known for CDR2 in kappa chains, hence, barring exceptional subject antibody sequences, assignment is automatic.

For light chain CDR3, up to six canonical structure types have been described for CDR3 in kappa chains, but three of these are rare. The three common ones can be distinguished by their length, reflected in Kabat numbering of residue positions 91-97:
Canonical structure type 1: 91, 92, 93, 94, 95, 96, 97 (also with an obligatory Pro at position 95 and Gln, Asn, or His at position 90).
Canonical structure type 3: 91, 92, 93, 94, 95, 97.
Canonical structure type 5: 91, 92, 93, 94, 95, 96, 96a, 97.

After identifying the canonical CDR structure types of the subject non-human antibody, human genes of the same chain type (heavy or light) that have the same combination of canonical structure types as the subject antibody are identified to form a candidate set of human sequences. In preferred embodiments, only the peptide sequences of human germline immunoglobulin V_{H} and Vₖ gene fragments are considered for comparison. Most of these gene fragments have been discovered and have already been assigned to a canonical structure type (Chothia, C., Lesk, A. M., Gherardi, E., Tomlinson, I. M., Walter, G., Marks, J. D., Llewelyn, M. B. & Winter, G. (1992) Structural repertoire of the human V_{H} segments. J. Mol. Biol. 227, 799-817; Tomlinson, I. M., Cox, J. P. L., Gherardi, E., Lesk, A. M. & Chothia, C. (1995) The structural repertoire of the human Vₖ domain. EMBO J. 14, 4628-4638). Additional V gene fragments not disclosed by these references are provided herein and appear among those sequences listed in FIG. 1 and FIG. 4. For the heavy chain, conformity of CDR1 and CDR2 to the mouse canonical structure types is assessed, and genes that do not conform are excluded. For the light chain, conformity of CDR1 and CDR2 of each human sequence to the canonical structure types of the subject antibody is first assessed. The potential of residues 89-95 of a candidate Vₖ gene to form a CDR3 of the same canonical structure type as the subject antibody is assessed, by positing a fusion of the gene with a J region and applying criteria for CDR3 canonical CDR structure type determination to the fused sequence, and non conforming sequences are excluded.

In another embodiment, appropriate when a variable domain of the subject antibody is of a canonical structure type not available in the human genome, human germline V genes that have three-dimensionally similar, but not identical, canonical structure types are considered for comparison. Such a circumstance often occurs with kappa chain CDR1 in murine antibodies. All 6 possible canonical structure types have been observed at this CDR in murine antibodies, whereas the human genome encodes only canonical types 2, 3, 4 and 6. In these circumstances, a canonical CDR structure type having length of amino acid residues within two of the length of the amino acid residues of the subject non-human sequence may selected for the comparison. For example, where a type 1 canonical structure is found in the subject antibody, human Vₖ sequences with canonical structure type 2 should be used for comparison. Where a type canonical structure is found in the murine antibody, human Vₖ sequences with either canonical structure type 3 or 4 should be used for comparison.

In another embodiment, mature, rearranged human antibody sequences can be considered for the sequence comparison. Such consideration might be warranted under a variety of circumstances, including but not limited to instances where the mature human sequence (1) is very close to germline; (2) is known not to be immunogenic in humans; or (3) contains a canonical structure type identical to that of the subject antibody, but not found in the human germline.

In preferred embodiments, for each of the candidate V genes with matching canonical structure types, residue-to-residue sequence identity and/or homology with the subject sequence is also evaluated to rank the candidate human sequences. In a specific embodiment, the residues evaluated are as follows:

| Chain | CDR | Residue positions |
|---|---|---|
| Kappa | 1 | 26-32 |
| Kappa | 2 | 50-52 |
| Kappa | 3 | 91-96 |
| Heavy | 1 | 31-35 |
| Heavy | 2 | 50-60 |

In preferred embodiments, residue-to-residue homology is first scored by the number of identical amino acid residues between the subject and the candidate human sequences. The human sequence used for subsequent construction of a converted antibody is chosen from among the 25 percent of candidates with the highest score. In other embodiments, appropriate when several candidate sequences have similar identity scores, similarity between non-identical amino acid residues may be additionally considered. Aliphatic-with-aliphatic, aromatic-with-aromatic, or polar-with-polar matches between subject and object residues are added to the scores. In another embodiment, quantitative evaluation of sequence homology may be performed using amino acid substitution matrices such as the BLOSUM62 matrix of Henikoff and Henikoff (Henikoff, S. & Henikoff, J. G. (1992) Amino acid substitution matrices from protein blocks. Proc. Natl. Acad. Sci. U.S.A 89, 10915-10919).

An object sequence for the framework region C-terminal to CDR3 sequence is selected from the set of known human germline J segments. A preferred J peptide sequence is selected by evaluating residue to residue homology for each J segment for sequence positions for which CDR3 and J overlap, using the scoring criteria specified for the evaluation of candidate V genes as mentioned above. The J gene segment peptide sequence used for subsequent construction of a converted antibody is chosen from among the 25 percent of candidates with the highest score. CDR3 of the heavy chain, which is part of the J_{H} region thereof, does not have a limited number of three-dimensional structures that can be predicted from its sequence, however, any J_{H} region may be used for constructing humanized heavy chain variable regions according to this method.

In one embodiment, the chimeric variable chain contains at least two CDRs from the subject non-human sequence, and framework sequences from the candidate human sequence. In other embodiments, a chimeric light chain contains three CDRs from the subject non-human sequence and framework sequences from the candidate human sequence. In other embodiments, a chimeric heavy chain contains at least two CDRs of the subject heavy chain, and framework sequence of the candidate human heavy chain. In another embodiment, a chimeric heavy chin contains each of the CDRs from the subject heavy chain and the framework sequences of the candidate human heavy chain. In still another embodiment, a chimeric antibody heavy chain contains CDRs 1 and 2 from the subject non-human sequence and residues 50-60 for CDR3 and residues 61-65 of a CDR from the candidate human heavy chain, along with the framework sequences of the candidate human sequence. In another embodiment, a chimeric heavy chain sequence contains each CDR from the subject non-human sequence, frameworks sequences 27-30 form the subject sequence, and the framework sequences from the candidate sequences. In all cases however, the chimeric antibody molecule contains no more than 10 amino acid residue in the framework sequence that differ from those in the framework sequence of the candidate human variable ration.

In another embodiment, appropriate when increased affinity of a humanized antibody is desired, residues within the CDRs of a converted antibody may be additionally substituted with other amino acids. Typically, no more than four amino acid residues in a CDR are changed, and most typically no more than two residues in the CDR will be changed, except for heavy chain CDR 2, where as many as 10 residues may be changed. Similarly, in certain embodiments, some of the amino acids in the framework sequences may be changed. In all embodiments, no more than 10 amino acid residues are changed.

The humanized antibody sequence is then physically assembled by methods of gene synthesis and recombinant protein expression known by those skilled in the art. The final form of the humanized sequences having the chimeric variable chains made by the methods disclosed herein may take many forms. Most typically, the chimeric antibodies will be made by construction a nucleic acid sequence encoding the chimeric variable chains, which are recombinantly expressed in a suitable cell type. Most typically, these variable regions will be linked to the constant regions of human immunoglobulin genes such that, when expressed, full-size immunoglobulins will be produced. In many cases, full-size IgG will be the preferred format. In other cases, IgG, IgM, IgA, IgD, or IgE may be preferred.

Functional equivalents also include single-chain antibody fragments, also known as single-chain antibodies (scFvs). These fragments contain at least one fragment of an antibody variable heavy-chain amino acid sequence (V_{H}) tethered to at least one fragment of an antibody variable light-chain sequence (V_{L}) with or without one or more interconnecting linkers. Such a linker may be a short, flexible peptide selected to assure that the proper three-dimensional folding of the (V_{L}) and (V_{H}) domains occurs once they are linked so as to maintain the target molecule binding-specificity of the whole antibody from which the single-chain antibody fragment is derived. Generally, the carboxyl terminus of the (V_{L}) or (V_{H}) sequence may be covalently linked by such a peptide linker to the amino acid terminus of a complementary (V_{L}) and (V_{H}) sequence. Single-chain antibody fragments may be generated by molecular cloning, antibody phage display library or similar techniques. These proteins may be produced either in eukaryotic cells or prokaryotic cells, including bacteria. ScFv's can also be fused to other parts if antibody molecules. For example, scFv's can be attached, via a natural or artificial peptide linker, to the CH2-CH3 region of an IgG to form a divalent scFv-Fc construct. Functional equivalents further include fragments of antibodies that have the same, or comparable binding characteristics to those of the whole antibody. Such fragments may contain one or both Fab fragments or the F(ab′)2 fragment. In most embodiments, the method will include screening candidate chimeric antibodies to select those having an association constant for the antigen suitable for an intended use. In most embodiments the humanized antibody made according to these methods will have an association constant for its antigen of 10⁶ M⁻¹, at least 10⁷ M⁻¹, at least 10⁸ M⁻¹ or at least 10⁹ M⁻¹.

“Epitope” refers to the region of an antigen that is contacted by an antibody. For example, for an antibody that binds to the F glycoprotein of RSV, only a portion of the surface area of that antigen will be contacted by the antibody upon formation of the antibody-antigen interaction. One way to define an epitope is to determine the structure of an antibody-antigen complex. To determine whether two different antibodies recognize identical or overlapping epitopes of an antigen, the structures of both antibody-antigen complexes may be elucidated and then compared. Because structure determination requires extensive experimentation, however, a simpler method for determining whether two antibodies recognize the same epitope is to determine whether they bind competitively to the antigen. According to this functional definition, if one antibody, when added in vast excess over the other antibody, reduces the binding of this other antibody to the antigen, then the two antibodies are deemed to recognize overlapping epitopes on the antigen.

The following Example illustrates the present invention by showing exemplary embodiments of humanized antibodies that bind RSV. One of ordinary skill in the art will understand that many other specific embodiments may be created using the methods disclosed herein, and that the present invention is not limited by the specific examples. For example, while the humanized antibodies described in the Example below were designed using the “super-humanizing” method described above and in co-pending U.S. patent application Ser. No. 10/194,975 and the continuation application filed therefrom on Feb. 8, 2005 (Attorney Docket No. 501231.13) to Foote, which does not select framework sequences from human variable regions based on framework similarity between the non-human antibody and the human variable regions, the CDR sequences can also be grafted into human framework sequences that are selected based on similarity to the non-human frameworks, such as described in U.S. Pat. Nos. 6,639,055, 6,423,511, 6,180,370, 6,054,297, 5,693,762, 5,693,761, 5,585,089, 5,530,101, 6,632,927, 5,859,205, 6,800,738, 6,719,971, 6,479,284, 6,407,213, 6,054,297, 5,795,965 and 5,225,539, each incorporated herein by reference.

In addition, although the invention is exemplified using the particular mouse monoclonal antibody HNK20, that happens to bind an epitope on the F glycoprotein of RSV, the invention can be practiced starting with any non-human antibody that binds any epitope of any protein of RSV, so long as the sequence of the variable regions of the non-human antibody is known so that the CDRs can be determined. Other non-human antibodies that bind RSV are described, for example, in U.S. Pat. Nos. 5,824,307 and 6,656,467, and in patent publications WO/0243660, WO/9605229 and WO/03063767, each incorporated herein by reference.

Further, although the humanized variable regions described below are made by grafting all three CDRs from each variable region of the non-human antibody, the invention can be practiced by grafting only two CDRs, because it has been demonstrated in U.S. Pat. No. 6,569,430 to Waldmann, et al., incorporated herein by reference, that epitope binding can be maintained when only two CDRs are grafted. Moreover, although particular sequences are presented below, mutations in the frameworks or CDRs of these particular sequences can be made, so longa as there are no more than 10 amino acid differences in the chimeric variable region. Such mutations may be executed to further mature the antibody for increased binding, stability or other purposes.

### EXAMPLE

The sequences of the subject variable domains come from the murine IgA antibody HNK20, which is described in U.S. Pat. Nos. 5,534,411 and 6,258,529, incorporated herein by reference in their entirety. This antibody has also been described in the published literature (Weltzin R, Hsu S A, Mittler E S, Georgakopoulos K, Monath T P (1994). Intranasal monoclonal immunoglobulin A against respiratory syncytial virus protects against upper and lower respiratory tract infections in mice. Antimicrob Agents Chemother 38(12):2785-91; Weltzin R, Traina-Dorge V, Soike K, Zhang J Y, Mack P, Soman G, Drabik G, Monath T P. (1996). Intranasal monoclonal IgA antibody to respiratory syncytial virus protects rhesus monkeys against upper and lower respiratory tract infection. J Infect Dis 174(2):256-61; Guirakhoo F, Catalan J, Monath T, Weltzin R. (1996) Cloning, expression and functional activities of a single chain antibody fragment directed to fusion protein of respiratory syncytial virus. Immunotechnology 2(3):219-28). The murine HNK20 binds to a region (epitope) of the F glycoprotein on RSV viral particles potently neutralizes it in vitro and in vivo, and has been tested in both animals and humans. For humanized variants of this or other RSV-binding antibodies, their utility will be a function of how effective they are at neutralizing the virus, thus preventing it from infecting human cells. In addition to being tested in its original form, as a murine IgA, a single-chain variable domain construct was also shown to bind and neutralize virus (Delagrave S, Catalan J, Sweet C, Drabik G, Henry A, Rees A, Monath T P, Guirakhoo F. (1999) Effects of humanization by variable domain resurfacing on the antiviral activity of a single-chain antibody against respiratory syncytial virus. Protein Eng 12(4):357-62). In an effort to reduce immune response in humans, these authors made 7-10 point mutations in the murine framework regions to make it more resemble a human variable domains. This method, which they refer to as “humanization” is not what is conventionally called humanization because it does not involve CDR-grafting. CDR-grafting of the CDRs from HNK20 onto human variable domain frameworks has not, to our knowledge, been attempted prior to the examples shown in the present invention.

As reported in U.S. Pat. No. 6,258,529B1, and in the manuscripts by Guirakhoo et al (Guirakhoo F, Catalan J, Monath T, Weltzin R. (1996) Cloning, expression and functional activities of a single chain antibody fragment directed to fusion protein of respiratory syncytial virus. Immunotechnology 2(3):219-28) and by Delagrave et al (Delagrave S, Catalan J, Sweet C, Drabik G, Henry A, Rees A, Monath T P, Guirakhoo F. (1999) Effects of humanization by variable domain resurfacing on the antiviral activity of a single-chain antibody against respiratory syncytial virus. Protein Eng 12(4):357-62) the sequence of the heavy and light chain variable regions of HNK20, referred to as SEQ ID NO. 1 and 2, are as follows:

SEQ ID NO. 1

HNK20 V_{H} (variable domain heavy chain):
EVQLQQSGAELVRPGALVKLSCKASGFNIKDYYMYWVKQRPEQGLEWIGW
IDPENGNTVYDPKFQGKASITADTSSNTAYLQLSSLASEDTAVYYCAYYG
TSYWFPYWGQGTLVTVSA:

SEQ ID NO. 2

HNK20 VL (variable domain light chain):
DIKVTQSPSSMYASLGERVTITCKASQDINNYLNWFQQKPGKSPKTLIYR
ANRLLDGVPSRFSGSGSGQDYSLTISSLEYEDMGIYYCLQFDEFPYTFGG
GTKLEIKR:

These are numbered according to Kabat as follows:

```
HNK20 V_{H}
                  1 2 3 4 5 6
1...•....0....•....0....•....0....•....0....•....0..a..•....0....•
QVQLQQSGAELVRPGALVKLSCKASGFNIKDYYMYWVKQRPEQGLEWIGWIDPENGNTVYDPKFQG
                                                                          1 1
        7 8 9 0 1
....0....•....0.2abc3.•....0....•....0a....•....0...
KASITADTSSNTAYLQLSSLASEDTAVYYCAYYGTSYWFPYWGQGTLVTVSA
```

Kabat-defined CDRs are underlined.

Canonical structure assignments according to Chothia for the V_{H} chain are as follows:
CDR1: Canonical structure 1 because of length (5 amino acids, numbered from 31 to 35).
CDR2: Limited to canonical structures 2 or 3 because of length (17 amino acids). Canonical structure 2 has P or S at 52a; G or S at 55, same as residues found here. Therefore this antibody has canonical structure 2.
CDR3: There are: no canonical structures for this position.

The sequence of the mouse variable chain with the CDRs defined by Kabat being identified by underlining is as follows:

```
HNK20 VL (Kappa)
                  1 2 3 4 5
1...•....0....•....0....•....0....•....0....•....0....•.
DIKVTQSPSSMYASLGERVTITCKASQDINNYLNWFQQKPGKSPKTLIYRANRLLD
                                                                                      1
      6 7 8 9 0
...0....•....0....•....0....•....0....•....0....•6a7
GVPSRFSGSGSGQDYSLTISSLEYEDMGIYYCLQFDEFPYTFGGGTKLEIKR
```

The canonical structure assignments according to Chothia for this Vₖ regions are as follows:
CDR1: Canonical structure 2, because of length (11 amino acids).
CDR2: Canonical structure 1, because of length (7 residues).
CDR3: Canonical structure 1, because of length and the presence of P at position 95 and Q, N, or H at position 90.

The HNK20 heavy chain has canonical structures 1 and 2 at CDR1 and CDR2, respectively, as determined using the rules mentioned above. FIG. 1 shows an alignment of human heavy chain germline variable gene-encoded amino acid sequences with canonical structures 1 at CDR1 and 2 at CDR2. Numbering is according to Kabat. Any of these sequences could be used as object sequences for grafting in the CDRs of the murine HNK20 heavy chain. They are shown in this figure aligned to the CDRs of the HNK20 heavy chain variable region.

To rank the selected human variable regions to determine which would be especially preferred for grafting of the murine CDRs, we compared the sequence identities in the CDRs of the human amino acid sequences to corresponding positions in the murine HNK20 CDRs that are likely to be in contact with antigen according to observed CDR-antigen interactions in other antibody-antigen complexes. As described above, CDR positions that are likely to be in contact with antigen, and that can be used to guide selection of human frameworks, are as follows (Kabat numbering):
CDR1: 31-35
CDR2: 50-60

In FIG. 1, these regions are underlined for the HNK20 CDRs. Sequence identities to these positions at the corresponding positions in the human germline sequences are underlined for the human heavy chain germline variable gene-encoded amino acid sequences. The number of amino acid matches between the HNK20 CDRs in the specified (underlined) regions and the corresponding positions in the human heavy chain germline variable gene-encoded amino acid sequences is shown at the right of each human sequence under the heading “#”. From this analysis, three human germline variable gene sequences (1-18*01, 1-24*01 and 7-4-1*01) are found to be the most preferred sequences since they have the highest sequence identity in these specified CDR regions (8, 7 and 7 matches, respectively).

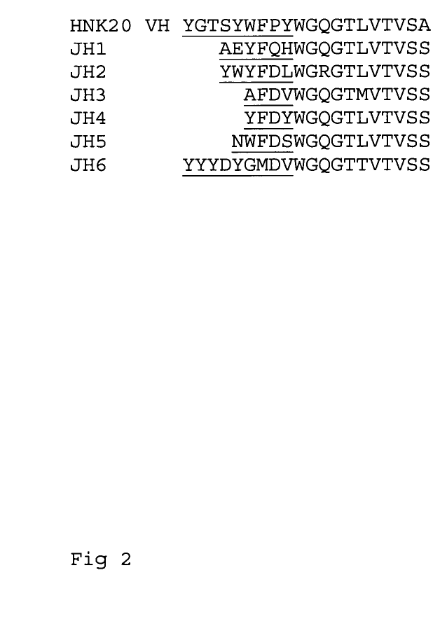

In addition to choosing human gemmline variable region sequences, the JH portions of the variable regions also must be chosen. JH sequences that may be used are shown in FIG. 2, aligned to the corresponding region of HNK20. The CDRs are underlined. Because canonical structures are not defined for CDR3 of the heavy chain, any of these may be used to build the human V region framework in the humanized antibodies.

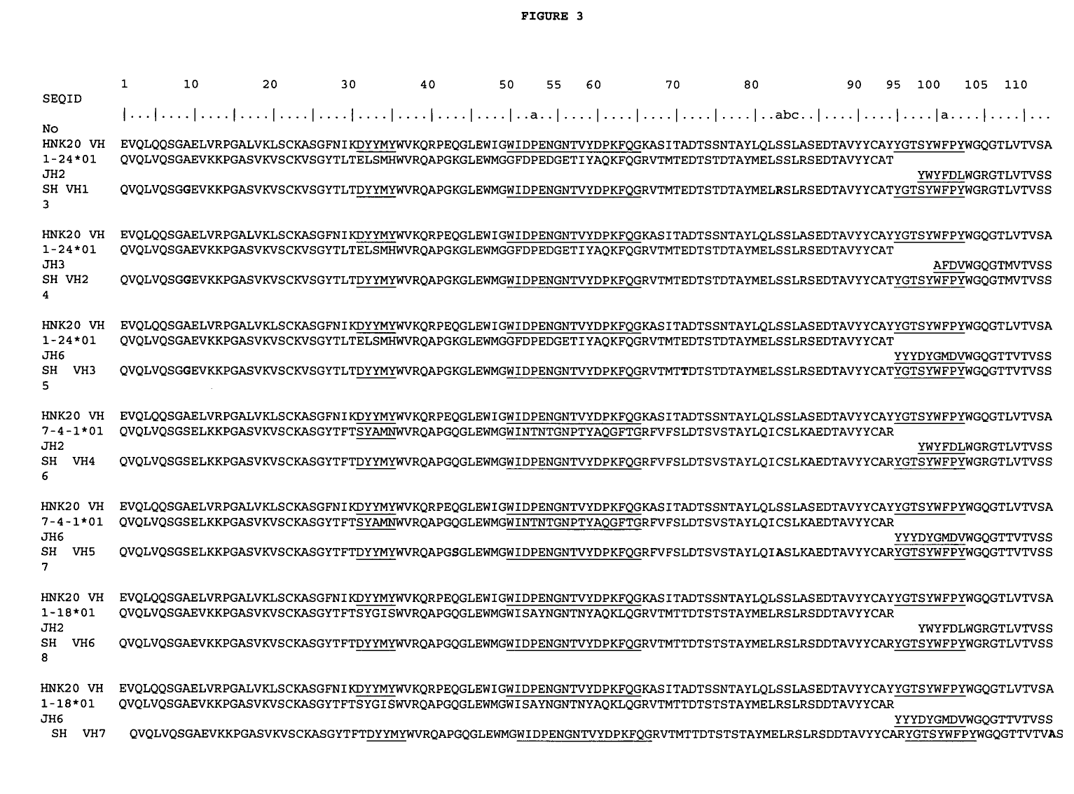

We provide examples of sequences of humanized heavy chain variable regions based on HNK20. For each of the most preferred human germline heavy chain variable gene sequences (1-18*01, 1-24*01 and 7-4-1*01), we show how the CDRs of HNK20 may be grafted onto these frameworks, including the region from the JH segments. FIG. 3 shows an alignment of HNK20 with the abovementioned candidate human germline variable gene-encoded amino acid segments. Numbering is according to Kabat and CDRs are underlined. Superhumanized examples are named SH V_{H}1, SH V_{H}2 . . . SH V_{H}7). SEQ ID NOS are shown at the right of each example of a humanized sequence. In some examples, a small number of amino acid changes in the framework regions away from the natural germline sequence have been introduced, in order to increase the predicted stability of the molecule by replacing natural amino acids with those that have a higher likelihood of forming the correct secondary structure (i.e. beta strands), or for other reasons. These changed amino acids are shown in bold-face type. One example is in the super-humanized example SH V_{H}5, in which the cysteine (C) present in the 7-4-1*01 human germline sequence was mutated to an alanine (A) in order to eliminate the chance of this residue oxidizing to form intermolecular or intramolecular disulfide bonds with other cysteines.

It is notable that the examples of super humanized antibodies have a low sequence identity with the donor murine antibodies in the framework regions. All of the examples of super humanized heavy chain V regions shown in FIG. 3 have less than 70% sequence identity with the donor antibody in the framework regions, and most examples (SH V_{H}1, SH V_{H}2, SH V_{H}3, SH V_{H}5 and SH V_{H}7) have below 65% sequence identity. This is in contrast to other humanization methods, such as those taught by Queen et al (U.S. Pat. No. 5,693,761), which result in humanized antibodies that have high sequence identity between the donor and humanized antibody frameworks.

FIG. 4 shows an alignment of human light (kappa) chain gemmine variable gene-encoded amino acid sequences with canonical structures 2, 1 and 1 for CDR1, CDR2 and CDR3, respectively, as discussed above. Numbering is according to Kabat. Any of these sequences could be used as object sequences for grafting in the CDRs of the murine HNK20 light chain. They are shown in this Figure aligned to the CDRs of the HNK20 kappa chain variable region. To determine which of these variable region genes are the most preferred for grafting the murine CDRs into, we compared the sequence identities in the CDRs of the human amino acid sequences to corresponding positions in the murine HNK20 CDRs that are likely to be in contact with antigen according to observed CDR-antigen interactions in other antibody-antigen complexes (as described above). These CDR positions that are likely to be in contact with antigen, and that can be used to guide selection of human frameworks, are as follows (Kabat numbering):
CDR1: 26-32
CDR2: 50-52
CDR3: 91-96

In FIG. 4, the regions of the CDRs that are considered for determining sequence identities with the human light chain germline variable genes are underlined for the donor Vₖ light chain from HNK20. Sequence identities to these positions are underlined for the human heavy chain germline variable gene-encoded amino acid sequences. The number of amino acid matches between the HNK20 CDRs in the specified (underlined) region and the corresponding positions in the human light (kappa) chain germline variable gene-encoded amino acid sequences is shown at the right of each sequence under the heading “#”.

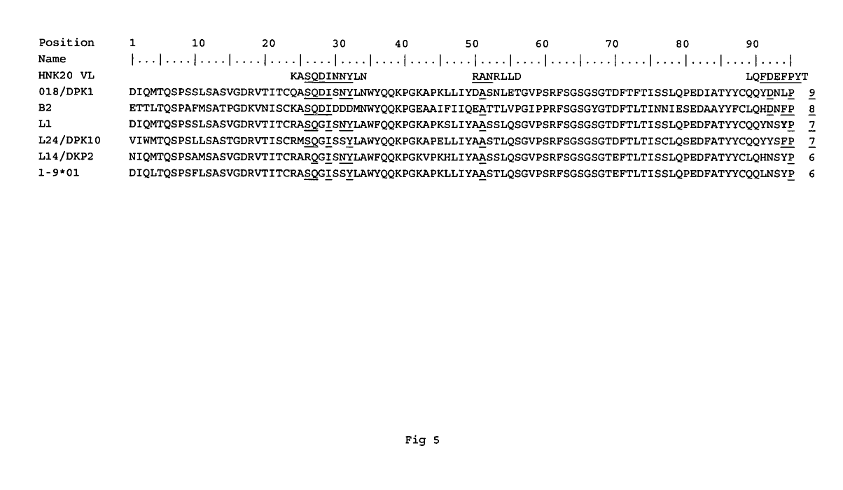

From this analysis, six human germline variable Vₖ gene sequences (018/DPK1, B2, L1, L24/DPK10, L14/DPK2 and 1-9*01) were ranked as before to determine the most preferred examples for CDR grafting. These sequences, aligned to the murine HNK20 Vₖ CDRs, are shown in FIG. 5.

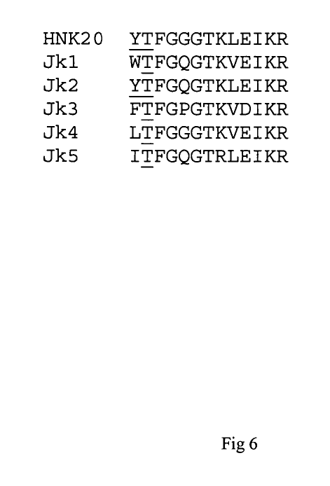

In addition to choosing human germline variable region sequences, Jkappa (Jk) sequences also must be chosen. The Jk sequences that may be used are shown in FIG. 6, aligned to the corresponding region of murine HNK20. CDR residues are underlined for HNK20. For the human germline Jk region-encoded amino acid sequences, the underlining indicates sequence identity in the CDR3 region with the donor amino acids. Although any of these may be used as a source of a human framework sequence for humanizing the HNK20 antibody, the segment Jk2 is the most preferred sequence since there is a perfect match in the CDR3 region to the donor sequence.

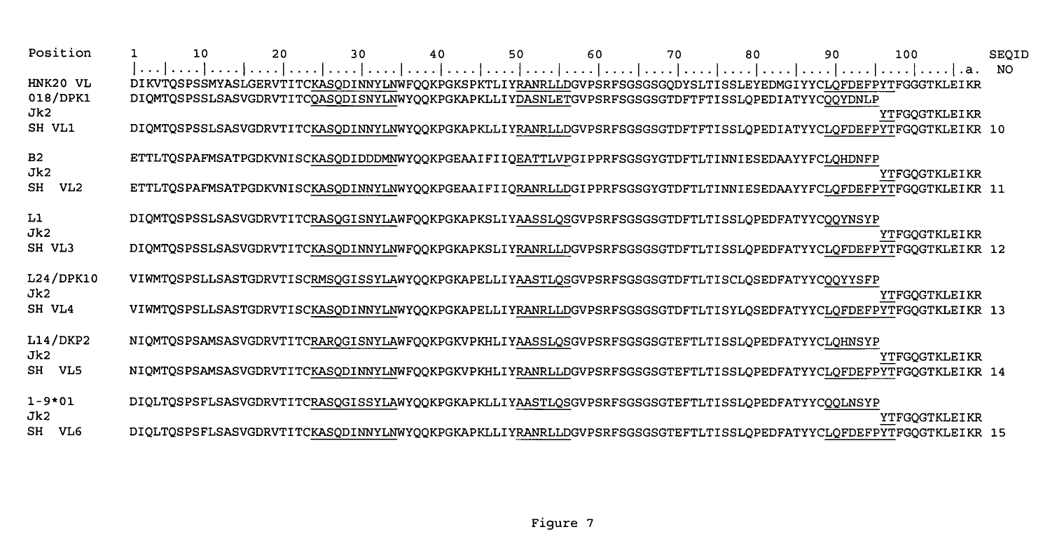

In FIG. 7, we provide examples of sequences of humanized Vₖ sequences based on HNK20. For each of these human germline variable gene-encoded amino acid sequences shown in FIG. 5, we show how the CDRs of HNK20 may be grafted onto these frameworks to create a humanized Vₖ. In this Figure, numbering is according to Kabat and CDRs are underlined. The humanized sequences are called SH V_{L}1, SH V_{L}2. SH V_{L}6. SEQ ID NOS are shown at the right of each example of a humanized sequence.

Typically, to reconstitute a functional, RSV-binding antibody or antibody fragment, it may be required to bring heavy chain and light chain variable region together. Any combination of the humanized heavy chain V_{H} and Vₖ regions provided herein may be combined to achieve this. One preferred combination is SH V_{H}7 and SH V_{L}1. These may be brought together in the context of a full-length antibody, a Fab, Fab′ or F(ab)′₂ fragment, and single-chain variable domain fragment, or any other antibody constructs known to one skilled in the art.

From the foregoing it will be appreciated that, although specific embodiments of the invention have been described herein for purposes of illustration, various modifications may be made without deviating from the spirit and scope of the invention. Accordingly, the invention is not limited except as by the following claims.

SEQUENCES:

SEQ ID NO. 1

HNX20 V_{H} (variable domain heavy chain):
EVQLQQSGAELVRPGALVKLSCKASGFNIKDYYMYWVKQRPEQGLEWIGW
IDPENGNTVYDPKFQGKASITADTSSNTAYLQLSSLASEDTAVYYCAYYG
TSYWFPYWGQGTLVTVSA

SEQ ID NO. 2

HNK20 VL (variable domain light chain):
DIKVTQSPSSMYASLGERVTITCKASQDINNYLNWFQQKPGKSPKTLIYR
ANRLLDGVPSRFSGSGSGQDYSLTISSLEYEDMGIYYCLQFDEFPYTFGG
GTKLEIKR

SEQ ID NO. 3

(super humanized heavy chain based on HNK20,
called SH V_{H}1):
QVQLVQSGGEVKKPGASVKVSCKVSGYTLTDYYMYWVRQAPGKGLEWMGW
IDPENGNTVYDPKFQGRVTMTEDTSTDTAYMELRSLRSEDTAVYYCATYG
TSYWFPYWGRGTLVTVSS

SEQ ID NO. 4

(super humanized heavy chain based on HNK20,
called SH V_{H}2):
QVQLVQSGGEVKKPGASVKVSCKVSGYTLTDYYMYWVRQAPGKGLEWMGW
IDPENGNTVYDPKFQGRVTMTEDTSTDTAYMELSSLRSEDTAVYYCATYG
TSYWFPYWGQGTMVTVSS

SEQ ID NO. 5

(super humanized heavy chain based on KNK20,
called SH V_{H}3):
QVQLVQSGGEVKKPGASVKVSCKVSGYTLTDYYMYWVRQAPGKGLEWMGW
IDPENGNTVYDPKFQGRVTMTTDTSTDTAYMELSSLRSEDTAVYYCATYG
TSYWFPYWGQGTTVTVSS

SEQ ID NO. 6

(super humanized heavy chain based on HNK20,
called SH V_{H}4):
QVQLVQSGSELKKPGASVKVSCKASGYTFTDYYMYWVRQAPGQGLEWMGW
IDPENGNTVYDPKFQGRFVFSLDTSVSTAYLQICSLKAEDTAVYYCARYG
TSYWFPYWGRGTLVTVSS

SEQ ID NO. 7

(super humanized heavy chain based on HNK20,
called SH V_{H}5):
QVQLVQSGSELKKPGASVKVSCKASGYTFTDYYMYWVRQAPGSGLEWMGW
IDPENGNTVYDPKFQGRFVFSLDTSVSTAYLQIASLKAEDTAVYYCARYG
TSYWFPYWGQGTTVTVSS

SEQ ID NO. 8

(super humanized heavy chain based on HNK20,
called SH V_{H}6):
QVQLVQSGAEVKKPGASVKVSCKASGYTFTDYYMYWVRQAPGQGLEWMGW
IDPENGNTVYDPKFQGRVTMTTDTSTSTAYMELRSLRSDDTAVYYCARYG
TSYWFPYWGRGTLVTVSS

SEQ ID NO. 9

(super humanized heavy chain based on HNK20,
called SH V_{H}7):
QVQLVQSGAEVKKPGASVKVSCKASGYTFTDYYMYWVRQAPGQGLEWMGW
IDPENGNTVYDPKFQGRVTMTTDTSTSTAYMELRSLRSDDTAVYYCARYG
TSYWFPYWGQGTTVTVAS

SEQ ID NO. 10

(super humanized kappa light chain based on HNK20,
called SH V_{L}1):
DIQMTQSPSSLSASVGDRVTITCKASQDINNYLNWYQQKYPGKAPKLLIY
RANRLLDGVPSRFSGSGSGTDFTFTISSLQPEDIATYYCLQFDEFPYTFG
QGTKLEIKR

SEQ ID NO. 11

(super humanized kappa light chain based on HNK20,
called SH V_{L}2):
ETTLTQSPAFMSATPGDKVNISCKASQDINNYLNWYQQKPGEAAIFIIQR
ANRLLDGIPPRFSGSGYGTDFTLTINNIESEDAAYYFCLQFDEFPYTFGQ
GTKLEIKR

SEQ ID NO. 12

(super humanized kappa light chain based on HNK20,
called SH V_{L}3):
DIQMTQSPSSLSASVGDRVTITCKASQDINNYLNWFQQKPGKAPKSLIYR
ANRLLDGVPSRFSGSGSGTDFTLTISSLQPEDFATYYCLQFDEFPYTFGQ
GTKLEIKR

SEQ ID NO. 13

(super humanized kappa light chain based on HNK20,
called SH V_{L}4):
VIWMTQSPSLLSASTGDRVTISCKASQDINNYLNWYQQKPGKAPELLIYR
ANRLLDGVPSRFSGSGSGTDFTLTISYLQSEDFATYYCLQFDEFPYTFGQ
GTKLEIKR

SEQ ID NO. 14

(super humanized kappa light chain based on HNK20,
called SH V_{L}5):
NIQMTQSPSAMSASVGDRVTITCKASQDINNYLNWFQQKPGKVPKHLIYR
ANRLLDGVPSRFSGSGSGTEFTLTISSLQPEDFATYYCLQFDEFPYTFGQ
GTKLEIKR

SEQ ID NO. 15

(super humanized kappa light chain based on HNK20,
called SH V_{L}6):
DIQLTQSPSFLSASVGDRVTITCKASQDINNYLNWYQQKPGKAPKLLIYR
ANRLLDGVPSRFSGSGSGTEFTLTISSLQPEDFATYYCLQFDEFPYTFGQ
GTKLEIKR

### REFERENCES

Al-Lazikani B, Lesk A M, Chothia C. (1997). Standard conformations for the canonical structures of immunoglobulins. J Mol. Biol. 273(4):927-48.
Anderson L J, Bingham P, Hierholzer J C. (1988). Neutralization of respiratory syncytial virus by individual and mixtures of F and G protein monoclonal antibodies. J Virol6 2(11):4232-8.
Carter, P., Kelley, R. F., Rodrigues, M. L., Snedecor, B., Covarrubias, M., Velligan, M. D., Wong, W. L. T., Rowland, A. M., Kotts, C. E., Carver, M. E., Yang, M., Bourell, J. H., Shepard, H. M. & Henner, D. (1992) High level Escherichia coli expression and production of a bivalent humanized antibody fragment. Bio/Technology 10, 163-167.
Chothia, C. & Lesk, A. M. (1987) Canonical structure types for the hypervariable regions of immunoglobulins. J. Mol. Biol. 96, 901-917.
Chothia, C., Lesk, A. M., Gherardi, E., Tomlinson, I. M., Walter, G., Marks, J. D., Llewelyn, M. B. & Winter, G. (1992) Structural repertoire of the human V_{H} segments. J. Mol. Biol. 227, 799-817.
Chomczynski, P. & Sacchi, N. (1987) Single-step method of RNA isolation by acid guanidinium thiocyanate-phenol-chloroform extraction. Anal. Biochem. 162, 156-159.
Delagrave S, Catalan J, Sweet C, Drabik G, Henry A, Rees A, Monath T P, Guirakhoo F. (1999) Effects of humanization by variable domain resurfacing on the antiviral activity of a single-chain antibody against respiratory syncytial virus. Protein Eng 12(4):357-62.
Foote, J. & Winter, G. (1992) Antibody residues affecting conformation of the hypervariable loops. J. Mol. Biol. 224, 487-499.
Guirakhoo F, Catalan J, Monath T, Weltzin R. (1996) Cloning, expression and functional activities of a single chain antibody fragment directed to fusion protein of respiratory syncytial virus. Immunotechnology 2(3):219-28.
Hansen, J. A., Martin, P. J. & Nowinski, R. C. (1980) Monoclonal antibodies identifying a novel T cell antigen and Ia antigens of human lymphocytes. hnmunogenetics 10, 247-260.
Henikoff, S. & Henikoff, J. G. (1992) Amino acid substitution matrices from protein blocks. Proc. Natl. Acad. Sci. U.S.A 89, 10915-10919.
Jin, L., Fendly, B. M. & Wells, J. A. (1992) High resolution functional analysis of antibody-antigen interactions. J. Mol. Biol. 226, 851-865.
Jones, P. T., Dear, P. H., Foote, J., Neuberger, M. S. & Winter, G. (1986) Replacing the complementarity-determining regions in a human antibody with those from a mouse. Nature 321, 522-525.
Jonsson, U., Fgerstam, L., Ivarsson, B., Lundh, K., Lof.ang.s, S., Persson, B., Roos, H., Ronnberg, I., Sjolander, S., Stenber, E., St.ang.hlberg, R., Urbaniczky, C., stlin, H. & Malmqvist, M. (1991) Real-time biospecific interaction analysis using surface plasmon resonance and a sensor chip technology. BioTechniques 11, 620-627.
Kabat, E. A., Wu, T. T., Perry, H. M., Gottesman, K. S. & Coeller, K. (1991) Sequences of proteins of immunological interest. 5th ed. 1991, Bethesda: U.S. Dept. of Health and Human Services, PHS, NIH.
Licea, A. F., Becerril, B. & Possani, L. D. (1996) Fab fragments of the monoclonal antibody BCF2 are capable of neutralizing the whole soluble venom from the scorpion Centruroides noxius Hoffman. Toxicon 34, 843-847.
MacCallum, R. M., Martin, A. C. R. & Thornton, J. M. (1996) Antibody-antigen interactions: contact analysis and binding site topography. J. Mol. Biol. 262, 732-745.
Martin, A. C. R. & Thornton, J. M. (1996) Structural families in loops of homologous proteins: automatic classification, modelling and application to antibodies. J. Mol. Biol. 263, 800-815.
Padlan. E. (1991) A possible procedure for reducing the immunogenicity of antibody variable domains while preserving their ligand binding properties, Molecular Immunology 28:489-498.
Padlan, E. O., Abergel, C. & Tipper, J. P. (1995) Identification of specificity-determining residues in antibodies. FASEB J. 9, 133-139.
Riechmann, L., Clark, M., Waldmann, H. & Winter, G. (1988) Reshaping human antibodies for therapy. Nature 332, 323-327.
Rutgeerts, P., et al (1999) Efficacy and safety of retreatment with anti-tumor necrosis factor antibody (INFLIXIMAB) to maintain remission in Crohn's disease. Gastroenterology 117, 761-769.
Selisko, B., Licea, A. F., Becerril, B., Zamudio, F., Possani, L. D. & Honjales, E. (1999) Antibody BCF2 against scorpion toxin Cn2 from Centruroides noxius Hoffman: primary structure and three-dimensional model as free Fv fragment and complexed with its antigen. Proteins 37, 130-143.
Tamura, M., Milenic, D., Iwahashi, M., E., P., Schlom, J. & Kashmiri, S. (2000) Structural Correlates of an Anticarcinoma Antibody: Identification of Specificity-Determining Residues (SDRs) and Development of a Minimally Immunogenic Antibody Variant by Retention of SDRs Only. J. Immunol. 164, 1432-1441.
Tomlinson, I. M., Cox, J. P. L., Gherardi, E., Lesk, A. M. & Chothia, C. (1995) The structural repertoire of the human Vₖ domain. EMBO J. 14, 4628-4638.
Weltzin R, Hsu S A, Mittler E S, Georgakopoulos K, Monath T P (1994). Intranasal monoclonal immunoglobulin A against respiratory syncytial virus protects against upper and lower respiratory tract infections in mice. Antimicrob Agents Chemother 38(12):2785-91.
Weltzin R, Traina-Dorge V, Soike K, Zhang J Y, Mack P, Soman G, Drabik G, Monath T P. (1996). Intranasal monoclonal IgA antibody to respiratory syncytial virus protects rhesus monkeys against upper and lower respiratory tract infection. J Infect Dis 174(2):256-61.
Wu, T. T. & Kabat, E. A. (1970) An analysis of the sequences of the variable regions of Bence Jones proteins and myeloma light chains and their implications for antibody complementarity. J. Exp. Med. 132, 211-250.
Ye, Q.-Z., Johnson, L. L. & Baragi, V. (1992) Gene synthesis and expression in E. coli for PUMP, a human matrix metalloproteinase. Biochem. Biophys. Res. Comm. 186,143-149.

1. A humanized antibody that binds an epitope of RSV, comprising
at least two CDRs from a non-human antibody variable region of a non-human antibody grafted to framework sequences from a human antibody variable region to form a chimeric variable region, where the humanized antibody competitively binds to an epitope that is bound by the mouse HNK20 antibody.

2. The humanized antibody of claim 1 wherein the humanized antibody comprises a chimeric heavy chain variable region.

3. The humanized antibody of claim 1 wherein the humanized antibody comprises a chimeric light chain variable region.

4. The humanized antibody of claim 1 wherein the humanized antibody comprises a chimeric light chain and a chimeric heavy chain variable region.

5. The humanized antibody of claim 1 wherein each of 3 CDRs from the non-human variable region are grafted to the variable region human framework sequences.

6. The humanized antibody of claim 1 wherein each of 3 CDRs from the non-human variable region light chain and heavy chain are grafted to the light and heavy chain framework sequences from the human antibody variable regions.

7. The humanized antibody of claim 1 further comprising a human antibody constant region.

8. The humanized antibody of claim 1 wherein the non-human variable region is a mouse or rat variable region.

9. The humanized antibody of claim 1 wherein the human variable region sequence is selected from the group consisting of Vₖ, V_{λ}., V_{H}, J_{H}, Jₖ and J_{λ} sequences.

10. The humanized antibody of claim 1 wherein chimeric variable light chains and chimeric heavy chains are assembled to form a molecule selected from the group consisting of a Fab fragment, a (Fab)₂ molecule, and a single chain Fv molecule.

11. The humanized antibody of claim 1 wherein the human variable region sequence is a sequence from a human germline variable region fragment.

12. The humanized antibody of claim 1 wherein the humanized antibody has an association constant for its antigen of at least 10⁶ M⁻¹, at least 10⁷ M⁻¹, at least 10⁸ M⁻¹ or at least 10⁹ M⁻¹.

13. The humanized antibody of claim 1 wherein the humanized antibody does not elicit an immune response when administered to a human.

14. The humanized antibody of claim 1 wherein the non-human variable region CDR sequences comprise CDRs from the murine heavy chain of HNK20 according to SEQ ID NO: 1.

15. The humanized antibody of claim 1 wherein the subject variable region CDR sequences comprise CDRs from the murine light chain of HNK20 according to SEQ ID NO:2.

16. The humanized antibody of claim 1 wherein the chimeric variable region comprises any of the sequences selected from the group consisting of SEQ ID NOS:3-9.

17. The humanized antibody of claim 1 wherein the chimeric variable region comprises any of the sequences selected from the group consisting of SEQ ID NOS:10-15.

18. The humanized antibody of claim 1 wherein the chimeric variable region comprises the heavy chain variable region sequence of SEQ ID NO:3 and light chain variable region sequence of SEQ ID NO:6.

19. The humanized antibody of claim 16 wherein the molecule binds RSV with an association constant of at least 10⁸ M⁻¹, at least 10⁹ M⁻¹ or at least 10¹⁰ M⁻¹.

20. The humanized antibody of claim 1 wherein the humanized antibody is an IgG1 antibody.

21. A nucleic acid sequence encoding the humanized antibody of claim 1.

22. A vector operably configured with control sequences to express the nucleic acid sequence of claim 21 in a cell.

23. The humanized antibody of claim 1 that is made in a fungus by expression from a vector operably configured with control sequences to express a nucleic acid sequence encoding the antibody of claim 1 in the fungus.

24. A humanized antibody that binds to RSV comprising,
a chimeric antibody variable region containing at least two non-human CDR sequences grafted to human variable framework sequences, the human framework sequences in the humanized antibody being characterized by being selected for having human CDRs of the same canonical structure type as the non-human CDR sequences for at least two non-human CDRs, where the framework sequences of the humanized antibody differ by no more than 10 amino acids from the selected framework sequences in the human antibody variable region.

25. The humanized antibody of claim 24 characterized in that the framework sequences of the humanized antibody heavy chain variable region have less than 65% sequence identity to the heavy chain variable region framework sequences of the non-human antibody.

26. The humanized antibody of claim 24 wherein each of 3 CDRs from the non-human variable region are grafted to the framework sequences of the human variable region.

27. The humanized antibody of claim 24 where the selected human variable region is from a germline variable region antibody sequence.

28. The humanized antibody of claim 24 where the antibody binds to an identical or overlapping epitope to that of the HNK20 antibody.

29. The humanized antibody of claim 24 wherein the chimeric variable region comprises a light chain variable region selected from the group consisting of SEQ ID NOS:10-15.

30. The humanized antibody of claim 24 wherein the chimeric variable region comprises a heavy chain variable region selected from the group consisting of SEQ ID NOS:3-9.

31. The humanized antibody of claim 24 comprising a light chain variable region selected from the group consisting of SEQ ID NOS:10-15 and a heavy chain variable region selected from the group consisting of SEQ ID NOS:3-9.

32. An antibody of any one of claims 24 wherein the antibody reduces the ability of RSV to infect cells.

33. The antibody of claim 1 wherein the antibody reduces the ability of RSV to infect cells.


SEQUENCE LISTING (78 sequences)

SEQ ID NO: 1 - protein, 118 aa, Mus musculus
EVQLQQSGAELVRPGALVKLSCKASGFNIKDYYMYWVKQRPEQGLEWIGWIDPENGNTVYDPKFQGKASITADTSSNTAYLQLSSLASEDTAVYYCAYYGTSYWFPYWGQGTLVTVSA

SEQ ID NO: 2 - protein, 108 aa, Mus musculus
DIKVTQSPSSMYASLGERVTITCKASQDINNYLNWFQQKPGKSPKTLIYRANRLLDGVPSRFSGSGSGQDYSLTISSLEYEDMGIYYCLQFDEFPYTFGGGTKLEIKR

SEQ ID NO: 3 - protein, 118 aa, Homo sapiens
QVQLVQSGGEVKKPGASVKVSCKVSGYTLTDYYMYWVRQAPGKGLEWMGWIDPENGNTVYDPKFQGRVTMTEDTSTDTAYMELRSLRSEDTAVYYCATYGTSYWFPYWGRGTLVTVSS

SEQ ID NO: 4 - protein, 118 aa, Homo sapiens
QVQLVQSGGEVKKPGASVKVSCKVSGYTLTDYYMYWVRQAPGKGLEWMGWIDPENGNTVYDPKFQGRVTMTEDTSTDTAYMELSSLRSEDTAVYYCATYGTSYWFPYWGQGTMVTVSS

SEQ ID NO: 5 - protein, 118 aa, Homo sapiens
QVQLVQSGGEVKKPGASVKVSCKVSGYTLTDYYMYWVRQAPGKGLEWMGWIDPENGNTVYDPKFQGRVTMTTDTSTDTAYMELSSLRSEDTAVYYCATYGTSYWFPYWGQGTTVTVSS

SEQ ID NO: 6 - protein, 118 aa, Homo sapiens
QVQLVQSGSELKKPGASVKVSCKASGYTFTDYYMYWVRQAPGQGLEWMGWIDPENGNTVYDPKFQGRFVFSLDTSVSTAYLQICSLKAEDTAVYYCARYGTSYWFPYWGRGTLVTVSS

SEQ ID NO: 7 - protein, 118 aa, Homo sapiens
QVQLVQSGSELKKPGASVKVSCKASGYTFTDYYMYWVRQAPGSGLEWMGWIDPENGNTVYDPKFQGRFVFSLDTSVSTAYLQIASLKAEDTAVYYCARYGTSYWFPYWGQGTTVTVSS

SEQ ID NO: 8 - protein, 118 aa, Homo sapiens
QVQLVQSGAEVKKPGASVKVSCKASGYTFTDYYMYWVRQAPGQGLEWMGWIDPENGNTVYDPKFQGRVTMTTDTSTSTAYMELRSLRSDDTAVYYCARYGTSYWFPYWGRGTLVTVSS

SEQ ID NO: 9 - protein, 118 aa, Homo sapiens
QVQLVQSGAEVKKPGASVKVSCKASGYTFTDYYMYWVRQAPGQGLEWMGWIDPENGNTVYDPKFQGRVTMTTDTSTSTAYMELRSLRSDDTAVYYCARYGTSYWFPYWGQGTTVTVAS

SEQ ID NO: 10 - protein, 108 aa, Homo sapiens
DIQMTQSPSSLSASVGDRVTITCKASQDINNYLNWYQQKPGKAPKLLIYRANRLLDGVPSRFSGSGSGTDFTFTISSLQPEDIATYYCLQFDEFPYTFGQGTKLEIKR

SEQ ID NO: 11 - protein, 108 aa, Homo sapiens
ETTLTQSPAFMSATPGDKVNISCKASQDINNYLNWYQQKPGEAAIFIIQRANRLLDGIPPRFSGSGYGTDFTLTINNIESEDAAYYFCLQFDEFPYTFGQGTKLEIKR

SEQ ID NO: 12 - protein, 108 aa, Homo sapiens
DIQMTQSPSSLSASVGDRVTITCKASQDINNYLNWFQQKPGKAPKSLIYRANRLLDGVPSRFSGSGSGTDFTLTISSLQPEDFATYYCLQFDEFPYTFGQGTKLEIKR

SEQ ID NO: 13 - protein, 108 aa, Homo sapiens
VIWMTQSPSLLSASTGDRVTISCKASQDINNYLNWYQQKPGKAPELLIYRANRLLDGVPSRFSGSGSGTDFTLTISYLQSEDFATYYCLQFDEFPYTFGQGTKLEIKR

SEQ ID NO: 14 - protein, 108 aa, Homo sapiens
NIQMTQSPSAMSASVGDRVTITCKASQDINNYLNWFQQKPGKVPKHLIYRANRLLDGVPSRFSGSGSGTEFTLTISSLQPEDFATYYCLQFDEFPYTFGQGTKLEIKR

SEQ ID NO: 15 - protein, 108 aa, Homo sapiens
DIQLTQSPSFLSASVGDRVTITCKASQDINNYLNWYQQKPGKAPKLLIYRANRLLDGVPSRFSGSGSGTEFTLTISSLQPEDFATYYCLQFDEFPYTFGQGTKLEIKR

SEQ ID NO: 16 - protein, 118 aa, Homo sapiens
QVQLQQSGAELVRPGALVKLSCKASGFNIKDYYMYWVKQRPEQGLEWIGWIDPENGNTVYDPKFQGKASITADTSSNTAYLQLSSLASEDTAVYYCAYYGTSYWFPYWGQGTLVTVSA

SEQ ID NO: 17 - protein, 98 aa, Homo sapiens
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCAR

SEQ ID NO: 18 - protein, 98 aa, Homo sapiens
QVQLVQSGAEVKKPGASVKVSCKVSGYTLTELSMHWVRQAPGKGLEWMGGFDPEDGETIYAQKFQGRVTMTEDTSTDTAYMELSSLRSEDTAVYYCAT

SEQ ID NO: 19 - protein, 98 aa, Homo sapiens
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCAR

SEQ ID NO: 20 - protein, 98 aa, Homo sapiens
QVQLVQSGAEVKKPGSSVKVSCKASGGTFSSYTISWVRQAPGQGLEWMGRIIPILGIANYAQKFQGRVTITADKSTSTAYMELSSLRSEDTAVYYCAR

SEQ ID NO: 21 - protein, 98 aa, Homo sapiens
QVQLVQSGAEVKKPGSSVKVSCKASGGTFSSYAISWVRQAPGQGLEWMGRIIPILGIANYAQKFQGRVTITADKSTSTAYMELSSLRSEDTAVYYCAR

SEQ ID NO: 22 - protein, 98 aa, Homo sapiens
QVQLVQSGAEVKKPGSSVKVSCKASGGTFSSYAISWVRQAPGQGLEWMGGIIPIFGTANYAQKFQGRVTITADKSTSTAYMELSSLRSEDTAVYYCAR

SEQ ID NO: 23 - protein, 98 aa, Homo sapiens
QVQLVQSGAEVKKPGSSVKVSCKASGGTFSSYAISWVRQAPGQGLEWMGRIIPILGTANYAQKFQGRVTITADESTSTAYMELSSLRSEDTAVYYCAR

SEQ ID NO: 24 - protein, 98 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCAR

SEQ ID NO: 25 - protein, 98 aa, Homo sapiens
QVQLVQSGSELKKPGASVKVSCKASGYTFTSYAMNWVRQAPGQGLEWMGWINTNTGNPTYAQGFTGRFVFSLDTSVSTAYLQICSLKAEDTAVYYCAR

SEQ ID NO: 26 - protein, 17 aa, Homo sapiens
AEYFQHWGQGTLVTVSS

SEQ ID NO: 27 - protein, 17 aa, Homo sapiens
YWYFDLWGRGTLVTVSS

SEQ ID NO: 28 - protein, 15 aa, Homo sapiens
AFDVWGQGTMVTVSS

SEQ ID NO: 29 - protein, 15 aa, Homo sapiens
YFDYWGQGTLVTVSS

SEQ ID NO: 30 - protein, 16 aa, Homo sapiens
NWFDSWGQGTLVTVSS

SEQ ID NO: 31 - protein, 20 aa, Homo sapiens
YYYDYGMDVWGQGTTVTVSS

SEQ ID NO: 32 - protein, 95 aa, Homo sapiens
ETTLTQSPAFMSATPGDKVNISCKASQDIDDDMNWYQQKPGEAAIFIIQEATTLVPGIPPRFSGSGYGTDFTLTINNIESEDAAYYFCLQHDNFP

SEQ ID NO: 33 - protein, 95 aa, Homo sapiens
DIQMTQSPSSLSASVGDRVTITCQASQDISNYLNWYQQKPGKAPKLLIYDASNLETGVPSRFSGSGSGTDFTFTISSLQPEDIATYYCQQYDNLP

SEQ ID NO: 34 - protein, 95 aa, Homo sapiens
NIQMTQSPSAMSASVGDRVTITCRARQGISNYLAWFQQKPGKVPKHLIYAASSLQSGVPSRFSGSGSGTEFTLTISSLQPEDFATYYCLQHNSYP

SEQ ID NO: 35 - protein, 95 aa, Homo sapiens
DIQMTQSPSSLSASVGDRVTITCRASQGISNYLAWFQQKPGKAPKSLIYAASSLQSGVPSRFSGSGSGTDFTLTISSLQPEDFATYYCQQYNSYP

SEQ ID NO: 36 - protein, 95 aa, Homo sapiens
DIQMTQSPSSLSASVGDRVTITCRASQSISSYLNWYQQKPGKAPKLLIYAASSLQSGVPSRFSGSGSGTDFTLTISSLQPEDFATYYCQQSYSTP

SEQ ID NO: 37 - protein, 95 aa, Homo sapiens; identical to SEQ ID NO: 36

SEQ ID NO: 38 - protein, 95 aa, Homo sapiens
DIQMTQSPSSLSASVGDRVTITCRASQGIRNDLGWYQQKPGKAPKRLIYAASSLQSGVPSRFSGSGSGTEFTLTISSLQPEDFATYYCLQHNSYP

SEQ ID NO: 39 - protein, 95 aa, Homo sapiens
DIQMTQSPSSLSASVGDRVTITCRARQGISSWLAWYQQKPEKAPKSLIYAASSLQSGVPSRFSGSGSGTDFTLTISSLQPEDFATYYCQQYNSYP

SEQ ID NO: 40 - protein, 95 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYP

SEQ ID NO: 41 - protein, 95 aa, Homo sapiens; identical to SEQ ID NO: 40

SEQ ID NO: 42 - protein, 95 aa, Homo sapiens
DIQMTQSPSSVSASVGDRVTITCRASQGISSWLAWYQQKPGKAPKLLIYAASSLQSGVPSRFSGSGSGTDFTLTISSLQPEDFATYYCQQANSFP

SEQ ID NO: 43 - protein, 95 aa, Homo sapiens; identical to SEQ ID NO: 42

SEQ ID NO: 44 - protein, 95 aa, Homo sapiens
DIQLTQSPSFLSASVGDRVTITCRASQGISSYLAWYQQKPGKAPKLLIYAASTLQSGVPSRFSGSGSGTEFTLTISSLQPEDFATYYCQQLNSYP

SEQ ID NO: 45 - protein, 95 aa, Homo sapiens
AIRMTQSPFSLSASVGDRVTITCWASQGISSYLAWYQQKPAKAPKLFIYYASSLQSGVPSRFSGSGSGTDYTLTISSLQPEDFATYYCQQYYSTP

SEQ ID NO: 46 - protein, 95 aa, Homo sapiens
AIRMTQSPSSFSASTGDRVTITCRASQGISSYLAWYQQKPGKAPKLLIYAASTLQSGVPSRFSGSGSGTDFTLTISCLQSEDFATYYCQQYYSYP

SEQ ID NO: 47 - protein, 95 aa, Homo sapiens
VIWMTQSPSLLSASTGDRVTISCRMSQGISSYLAWYQQKPGKAPELLIYAASTLQSGVPSRFSGSGSGTDFTLTISCLQSEDFATYYCQQYYSFP

SEQ ID NO: 48 - protein, 95 aa, Homo sapiens
AIQMTQSPSSLSASVGDRVTITCRASQGIRNDLGWYQQKPGKAPKLLIYAASSLQSGVPSRFSGSGSGTDFTLTISSLQPEDFATYYCLQDYNYP

SEQ ID NO: 49 - protein, 95 aa, Homo sapiens
DVVMTQSPAFLSVTPGEKVTITCQASEGIGNYLYWYQQKPDQAPKLLIKYASQSISGVPSRFSGSGSGTDFTFTISSLEAEDAATYYCQQGNKHP

SEQ ID NO: 50 - protein, 95 aa, Homo sapiens
EIVMTQSPATLSVSPGERATLSCRASQSVSSNLAWYQQKPGQAPRLLIYGASTRATGIPARFSGSGSGTEFTLTISSLQSEDFAVYYCQQYNNWP

SEQ ID NO: 51 - protein, 95 aa, Homo sapiens; identical to SEQ ID NO: 50

SEQ ID NO: 52 - protein, 95 aa, Homo sapiens
EIVLTQSPATLSLSPGERATLSCRASQSVSSYLAWYQQKPGQAPRLLIYDASNRATGIPARFSGSGSGTDFTLTISSLEPEDFAVYYCQQRSNWP

SEQ ID NO: 53 - protein, 95 aa, Homo sapiens
EIVLTQSPDFQSVTPKEKVTITCRASQSIGSSLHWYQQKPDQSPKLLIKYASQSFSGVPSRFSGSGSGTDFTLTINSLEAEDAATYYCHQSSSLP

SEQ ID NO: 54 - protein, 95 aa, Homo sapiens; identical to SEQ ID NO: 53

SEQ ID NO: 55 - protein, 95 aa, Homo sapiens; identical to SEQ ID NO: 48

SEQ ID NO: 56 - protein, 95 aa, Homo sapiens; identical to SEQ ID NO: 47

SEQ ID NO: 57 - protein, 95 aa, Homo sapiens; identical to SEQ ID NO: 44

SEQ ID NO: 58 - protein, 95 aa, Homo sapiens; identical to SEQ ID NO: 42

SEQ ID NO: 59 - protein, 95 aa, Homo sapiens
DIQMTQSPSSLSASVGDRVTITCRASQGISSWLAWYQQKPEKAPKSLIYAASSLQSGVPSRFSGSGSGTDFTLTISSLQPEDFATYYCQQYNSYP

SEQ ID NO: 60 - protein, 95 aa, Homo sapiens; identical to SEQ ID NO: 39

SEQ ID NO: 61 - protein, 95 aa, Homo sapiens; identical to SEQ ID NO: 38

SEQ ID NO: 62 - protein, 95 aa, Homo sapiens; identical to SEQ ID NO: 33

SEQ ID NO: 63 - protein, 95 aa, Homo sapiens; identical to SEQ ID NO: 36

SEQ ID NO: 64 - protein, 95 aa, Homo sapiens; identical to SEQ ID NO: 45

SEQ ID NO: 65 - protein, 95 aa, Homo sapiens; identical to SEQ ID NO: 52

SEQ ID NO: 66 - protein, 95 aa, Homo sapiens
EIVLTQSPATLSLSPGERATLSCRASQSVSSYLAWYQQKPGQAPRLLIYDASNRATGIPARFSGSGSGRDFTLTISSLEPEDFAVYYCQQRSNWP

SEQ ID NO: 67 - protein, 95 aa, Homo sapiens; identical to SEQ ID NO: 50

SEQ ID NO: 68 - protein, 95 aa, Homo sapiens; identical to SEQ ID NO: 33

SEQ ID NO: 69 - protein, 95 aa, Homo sapiens; identical to SEQ ID NO: 32

SEQ ID NO: 70 - protein, 95 aa, Homo sapiens; identical to SEQ ID NO: 35

SEQ ID NO: 71 - protein, 95 aa, Homo sapiens; identical to SEQ ID NO: 47

SEQ ID NO: 72 - protein, 95 aa, Homo sapiens; identical to SEQ ID NO: 34

SEQ ID NO: 73 - protein, 95 aa, Homo sapiens; identical to SEQ ID NO: 44

SEQ ID NO: 74 - protein, 13 aa, Homo sapiens
WTFGQGTKVEIKR

SEQ ID NO: 75 - protein, 13 aa, Homo sapiens
YTFGQGTKLEIKR

SEQ ID NO: 76 - protein, 13 aa, Homo sapiens
FTFGPGTKVDIKR

SEQ ID NO: 77 - protein, 13 aa, Homo sapiens
LTFGGGTKVEIKR

SEQ ID NO: 78 - protein, 13 aa, Homo sapiens
ITFGQGTRLEIKR